(19) United States
(12) Patent Application Publication
(10) Pub. No.: US 2019/0150463 A1
(43) Pub. Date: May 23, 2019

(54) COMPOSITION FOR CONTROLLING ACQUIRED IMMUNE FUNCTION SUPPRESSION DUE TO ANTI-INFLUENZA DRUG, AND PRODUCTION METHOD FOR SAME

(71) Applicants: Meiji Co., Ltd., Chuo-ku, Tokyo (JP); Tokushima University, Tokushima-shi, Tokushima (JP)

(72) Inventors: Hiroshi KIDO, Tokushima-shi, Tokushima (JP); Etsuhisa TAKAHASHI, Tokushima-shi, Tokushima (JP); Seiya MAKINO, Odawara-shi, Kanagawa (JP); Hiroshi KANO, Odawara-shi, Kanagawa (JP); Jun HENMI, Odawara-shi, Kanagawa (JP)

(21) Appl. No.: 16/317,684

(22) PCT Filed: Aug. 15, 2017
(86) PCT No.: PCT/JP2017/029355

§ 371 Date: Jan. 14, 2019

(30) Foreign Application Priority Data
Aug. 17, 2016 (JP) 2016-159824

(51) Int. Cl. A23C 9/123; A23L 33/135; A61K 35/20; A61P 31/16; A61P 37/04

(52) U.S. Cl. CPC A23C 9/123; A23L 33/135; A61P 37/04; A61P 31/16; A61K 35/20

(57) ABSTRACT

A composition for suppressing deterioration of an acquired immune function according to the present invention includes, as an active ingredient, lactic acid bacteria products from lactic acid bacteria of the genus Lactobacillus. This composition for suppressing deterioration of the acquired immune function suppresses deterioration of the acquired immune function due to the use of an anti-influenza drug. The lactic acid bacteria of the genus Lactobacillus is preferably a bulgaricus bacterium. For example, the lactic acid bacteria of the genus Lactobacillus may be Lactobacillus delbrueckii subsp. bulgaricus. In the production method according to the present invention, a milk raw material is supplied to lactic acid bacteria of the genus Lactobacillus in order to produce the composition for suppressing deterioration of the acquired immune function to suppress deterioration of the acquired immune function due to the use of an anti-influenza drug.

### TECHNICAL FIELD

The present invention relates to a composition for suppressing deterioration of an acquired immune function by use of an anti-influenza drug and a production method thereof.

### BACKGROUND ART

Influenza develops by infection with influenza virus. The infectability of influenza is very strong, and in Japan about 10 million people are infected each year. Also, influenza is epidemic, and thus infection spreads to many people in a short period once the epidemic starts. Among them, the elderly often become severe, causing complications such as pneumonia, and there is also a risk of death.

Currently, anti-influenza drugs such as oseltamivir (OSV), for example, are commonly used for treating influenza. It is said that administering anti-influenza drugs within 48 hours after onset inhibits the growth of influenza virus and shortens the duration of disease. However, administering anti-influenza drugs reduces an amount of antigens in a body and may cause deterioration of the function of immunity acquired by living organisms. Deterioration of the acquired immune function of the living body removes virus from the body and reduces a production amount of specific antibodies for preventing re-infection. Thus, it is regarded as a problem that influenza infected people who used OSV have a higher re-infection rate of influenza in the following season than infected people who did not use OSV.

### CITATION LIST

### Patent literature

Patent Document 1: Published Japanese Translation No. 2010-518151
Patent Document 2: Japanese Unexamined Patent Application Publication No. 2012-72113

Patent Document 3: International Publication No. 2012/133827

### DISCLOSURE OF INVENTION

### Technical Problem

In recent years, it has been confirmed that certain lactic acid bacteria are effective for the prevention or treatment of influenza infection. For example, Patent Document 1 discloses the use of a bacterial strain of Lactobacillus casei species to produce an orally administrable composition for increasing protection against influenza after vaccination. In addition, Patent Document 2 discloses an agent for preventing and/or treating influenza containing lactic acid bacteria belonging to Lactobacillus acidophilus as an active ingredient. In addition, Patent Document 3 discloses an anti-influenza virus composition containing Lactobacillus paracasei as an active ingredient.

However, any of the above-mentioned Patent Documents 1 to 3 do not disclose inhibition of the deterioration of the immune function against the virus infection after administration of the anti-influenza drug.

In response to the above, it is an object of the present invention to provide a composition capable of suppressing deterioration of an acquired immune function caused by administration of an anti-influenza drug and a method for producing the same.

### Solution to Problem

As a result of intensive investigations in view of the above problems, the inventors of the present application have found that administering anti-influenza drugs with lactic acid bacteria products by lactic acid bacteria of the genus Lactobacillus suppresses deterioration of immune function against influenza virus as compared with a case where anti-influenza drugs are administered alone; thus, the present invention has been completed.

A composition for suppressing deterioration of an acquired immune function according to a first aspect of the present invention contains a lactic acid bacterial product by lactic acid bacteria of the genus Lactobacillus as an active ingredient and suppresses deterioration of the acquired immune function due to use of an anti-influenza drug. In other words, in the composition for suppressing deterioration of the acquired immune function according to one aspect of the present invention, a product of lactic acid bacteria by lactic acid bacteria of the genus Lactobacillus is used as the composition itself or as one component thereof. Note that the term “composition” as used herein includes preparations of medicines, supplements, food additives, and the like, foods and drinks (excluding animals and plants themselves), and materials that can be ingested by animals (including humans), such as foods and drinks composition (including processed foods and drinks).

In the above-described composition for suppressing deterioration of the acquired immune function, it is preferable that the lactic acid bacteria of the genus Lactobacillus are classified as a subspecies bulgaricus.

In the above-described composition for suppressing deterioration of acquired immune function, the lactic acid bacteria of the genus Lactobacillus is preferably Lactobacillus delbrueckii subsp. bulgaricus.

It is preferable that the composition for suppressing deterioration of the acquired immune function is fermented milk.

The composition for suppressing deterioration of the acquired immune function may further have an infection inhibitory action against influenza virus.

In addition, a production method according to another aspect of the present invention is a method for producing a composition for suppressing deterioration of an acquired immune function for supplying a milk raw material to a lactic acid bacterium of the genus Lactobacillus and suppressing deterioration of the acquired immune function due to the use of an anti-influenza drug.

### Effect of the Invention

As described above, the composition for suppressing deterioration of the acquired immune function of the present invention contains the lactic acid bacterial product by lactic acid bacteria of the genus Lactobacillus as an active ingredient. Administering such a composition for suppressing the deterioration of the acquired immune function with an anti-influenza drug increases an amount of a specific antibody against influenza virus as compared with the case where the anti-influenza drug is administered alone. Thus, the composition for suppressing the deterioration of the acquired immune function of the present invention allows for suppressing the deterioration of the acquired immune function due to the use of the anti-influenza drug. In addition, with the production method of the present invention, it is possible to produce a composition for suppressing deterioration of acquired immune function to suppress deterioration of the acquired immune function due to the use of an anti-influenza drug.

### BRIEF DESCRIPTION OF THE DRAWINGS

FIG. 1 is a graph showing the results of Test 1 (amount of IgA in a lung lavage fluid) according to this example.

FIG. 2 is a graph showing the results (amount of IgG in serum) of Test 1 according to this example.

FIG. 3 is a graph showing the results of Test 2 according to this example.

### DESCRIPTION OF THE PREFERRED EMBODIMENTS

Hereinafter, the present invention will be described more specifically. However, the present invention should be not limited to this.

### (1) Composition for Suppressing Deterioration of Acquired Immune Function

The composition for suppressing deterioration of an acquired immune function according to the present invention contains a lactic acid bacterium product by lactic acid bacteria of the genus Lactobacillus as an active ingredient. Here, lactic acid bacteria products include lactic acid bacteria fermented products, lactic acid bacteria cultures, lactic acid bacteria metabolites and the like. The lactic acid bacteria fermented product is a result (including cultures and products) obtained after lactic acid fermentation using lactic acid bacteria. In addition, the lactic acid bacteria culture is a resultant material (including cultures and products) obtained by culturing lactic acid bacteria in the presence of a medium suitable for culturing lactic acid bacteria. The lactic acid bacteria metabolite is a resultant material (including a product) obtained by metabolism of lactic acid bacteria. Note that the lactic acid bacteria fermented product and the lactic acid bacteria culture may mean the same one, and in such a case, they can also be used interchangeably.

The lactic acid bacteria products may or may not contain lactic acid bacteria per se (including viable and killed bacteria). From the viewpoint of probiotics, fermented lactic acid bacteria containing viable bacteria are preferably used.

In addition, “lactic acid bacteria” is a generic term for microorganisms that assimilate glucose to produce lactic acid whose yield based on sugar is 50% or more; it is a coccus or a rod-shaped bacterium of a gram-positive bacterium as a physiological property, and has features of no mobility, no sporulation ability and catalase negativity. Lactic acid bacteria have been eaten in various places throughout the world via fermented milk and the like since ancient times, and are said to be microorganisms with significant high safety. Lactic acid bacteria are classified into multiple genera.

Further, the composition for suppressing deterioration of the acquired immune function according to the present invention contains a lactic acid bacterial product produced by lactobacillus of the genus Lactobacillus, which is classified into the genus Lactobacillus, as an active ingredient. In other words, the composition for suppressing deterioration of the acquired immune function according to the present invention contains at least one of a fermented product of lactic acid bacteria of the genus Lactobacillus, a culture of lactic acid bacteria of the genus Lactobacillus and a metabolite of lactic acid bacteria of the genus Lactobacillus as an active ingredient.

Examples of lactic acid bacteria of the genus Lactobacillus include Bulgaricus subspecies, Casei species, Acidophilus species, Plantarum species, and the like. Among these lactic acid bacteria of the genus Lactobacillus, in the present invention, it is preferable to use lactic acid bacteria (also referred to as Bulgaricus bacteria) classified as bulgaricus subspecies. Furthermore, among the lactic acid bacteria of the genus Lactobacillus, it is more preferable to use Lactobacillus delbrueckii subsp. bulgaricus.

More specifically, Lactobacillus delbrueckii subsp. bulgaricus contains Lactobacillus delbrueckii subsp. bulgaricus OLL 1073R-1 bacterium (Deposit number: FERM BP-10741) (hereinafter referred to as “Bulgaricus bacterium R-1 strain”), and the like.

The composition for suppressing the deterioration of the acquired immune function according to the present invention further preferably contains the lactic acid bacterial product of “Bulgaricus bacterium R-1 strain” among various Lactobacillus genus lactic acid bacteria as an active ingredient. Bulgaricus bacterium R-1 strain was deposited, on Feb. 22, 1999 (the day of deposit), to the International Patent Organism Depository of the National Institute of Advanced Industrial Science and Technology (IPOD, AIST) (central 6th, 1-1 Higashi 1-chome Tsukuba city, Ibaraki prefecture, Japan) as a deposit number FERM P-17227 (national deposit) and was transferred to an international deposit under the Budapest Treaty on Nov. 29, 2006 with a deposit number IPOD FERM BP-10741. Note that since NITE Patent Microorganisms Depositary, National Institute of Technology and Evaluation (IPOD, NITE) succeeded the patent microorganism deposit business from the International Patent Organism Depositary of the National Institute of Advanced Industrial Science and Technology, the Bulgaricus bacterium R-1 strain currently has been deposited to NITE Patent Microorganisms Depositary, National Institute of Technology and Evaluation (IPOD, NITE) (#122, 2-5-8 Kazusakamatari, Kisarazu-shi, Chiba prefecture, Japan) (Deposit number: FERM BP-10741).

It is preferable that the lactic acid bacteria product contained in the composition for suppressing deterioration of an acquired immune function of the present invention is a fermented product of lactic acid bacteria. The fermented product of lactic acid bacteria includes fermented products of lactic acid bacteria and processed products thereof, for example, a culture filtrate and culture supernatant liquid obtained by filtering, centrifuging or membrane separation, etc., of cultures (fermented product of lactic acid bacteria), concentrate obtained by concentrating culture filtrate, culture supernatant liquid or fermented product of lactic acid bacteria etc. with an evaporator or the like, a paste product thereof, a diluted product thereof, dried matter (freezing, heating, decompression etc.) thereof or the like. In preparing the treated product, one or more of the above-described treatment steps such as bacteria elimination treatment (e.g. filtration, centrifugation, membrane separation), precipitation, concentration, paste formation, dilution, drying and the like can be carried out in combination. Examples of the medium for lactic acid bacteria culture include skimmed milk powder medium, MRS medium and the like.

More specifically, examples of the lactic acid bacterium product contained in the composition for suppressing deterioration of the acquired immune function of the present invention include a lactic acid bacterium fermented product obtained by fermenting various base materials using Bulgaricus bacterium R-1 strain.

The base material used for fermentation may be any one as long as it can form an environment in which fermentation can occur as a result of growth or proliferation of Bulgaricus bacterium R-1 strain. The base material is, for example, a food material such as milk of a human or animal, vegetables, fruits, beans, cereals, or the like, and it may be a medium for growing or proliferating a microorganism or raw milk. The base material is preferably a food material that can be ingested as a food after fermentation; more specifically it is a raw milk or a medium that includes raw milk (unbaked milk), pasteurized milk, whole fat concentrated milk, whole milk powder, skimmed milk powder, defatted concentrated milk, milk protein concentrate (MPC), whey, whey powder, desalted whey, desalted whey powder, whey protein concentrate (WPC), whey protein isolate (WPI), α-lactalbumin, β-lactoglobulin, casein, sodium caseinate, calcium caseinate, cream, butter, soy milk, or the like, and it may be a raw milk or a medium containing the above-described food materials to which carbohydrate (including lactose), minerals, vitamins, and/or yeast extract are added.

Note that Bulgaricus bacterium R-1 strain is known to produce extracellular polysaccharide (EPS) as a metabolite. Thus, the composition for suppressing deterioration of the acquired immune function of the present invention may contain extracellular polysaccharide produced from Bulgaricus bacterium R-1 strain. In the present invention, for example, the lower limit of the intake amount of extracellular polysaccharide per day is 500 μg, preferably 1.0 mg, and more preferably 2.0 mg. The upper limit should not be particularly limited, but is, for example, 8.0 mg.

Various other fermentative bacteria, such as lactic acid bacteria other than Bulgaricus bacterium R-1 strain, Bacillus subtilis var. natto, and/or yeast, may be used together in fermentation. More specifically, thermophilus bacteria (Streptococcus thermophilus) used as starter bacteria in production of yoghurt and Bacillus subtilis var natto used for fermentation of natto can be used.

It is particularly preferable that the product of lactic acid bacteria is a milk fermented product or a milk culture of lactic acid bacteria. Examples of the milk fermented product or the milk culture include fermented milk. Here, “fermented milk” means material obtained by fermenting milk. “Fermented milk” includes, for example, “fermented milk”, “lactic acid bacteria beverage”, “milk beverage”, “natural cheese”, or the like as defined by the Ministerial Ordinance on Milk and Milk products Concerning Compositional Standards etc. (Ministerial Ordinance on Milk, etc.); however, it should not be limited to this. For example, fermented milk is “fermented milk” defined by the Ministerial Ordinance on Milk, etc.; that is, the fermented milk is a material provided in the form of a solid (hard type), paste (soft type), or liquid (drink type), which is obtained by fermenting, with lactic acid bacteria or yeast, milk such as raw milk, cow milk, special milk, raw goat milk, sterilized goat milk, cream sheep's milk, ingredient-adjusted milk, low fat milk and processed milk etc. or milk etc. containing non-fat milk solids whose amount contained therein is approximately equal to or greater than the amount contained in the above-described milk, or the fermented milk is a frozen material thereof. However, the fermented milk should not be limited to those.

In the fermented milk of the present invention, the concentration range of non-fat milk solids content is preferably a range from 4.0% to 12.0% inclusive, more preferably a range from 6.0% to 10.0% inclusive, and further more preferably a range from 7.0% to 9.0% inclusive, for example. The concentration of the milk fat component is, for example, preferably within a range from 0.2% to 4.0% inclusive, more preferably within a range from 0.3% to 3.0% inclusive, and further more preferably within a range from 0.4% to 2.0% inclusive, for example.

A typical example of fermented milk is yoghurt. Yoghurt, for example, includes plain yoghurt, hard yoghurt (set type yoghurt), soft yoghurt, drink yoghurt and the like.

In addition, when the composition for suppressing deterioration of the acquired immune function of the present invention is to be provided as fermented milk, it is preferable that it is provided in the form of one package whose amount is suitable for one ingestion. This allows a necessary amount of the active ingredient to be ingested properly and easily, thus enhancing usability. Here, “form of one package” includes all packaging forms. Examples of the packaging form include a container with a lid, a bottle with a cap, a small bag, a pouch, a tube and the like. In the present invention, it is possible to clarify its application(s) or usage(s) by adding description of the use, efficacy, ingestion method, etc. of the product to each package for packing one piece or to a package containing a plurality of packages each of which is for packing one piece, by providing a package in which a description stuff is contained, and/or by separately presenting a description stuff such as a pamphlet or the like.

In addition, the composition for suppressing deterioration of the acquired immune function of the present invention can be provided as a food or drink in a form other than fermented milk. Specific examples of such foods and drinks include cheese, soft drinks, gums, gummies, jelly, biscuits, and the like. However, the form of the food or drink should not be particularly limited.

Subsequently, the physiological activity of the composition for suppressing deterioration of the acquired immune function according to the present invention will be described. The composition for suppressing deterioration of the acquired immune function of the present invention suppresses deterioration in the acquired immune function due to the use of the anti-influenza drug.

As anti-influenza drugs, for example, oseltamivir (OSV), zanamivir, peramivir, laninamivir octanoate ester hydrate and the like are known. Any of these are anti-influenza drugs that suppress the growth of virus by inhibiting neuraminidase. It is known that such anti-influenza drugs can suppress the growth of viruses when administered at the time of influenza infection, and shorten the duration of disease, whereas they cause deterioration in acquired immune function against influenza virus infection. Thus, people to whom anti-influenza drugs have been administered are more likely to be re-infected with influenza after the following season than those to whom anti-influenza drugs have not been administered. This is due to the fact that administration of anti-influenza drug decreases the production of specific antibodies that prevent re-infection of the virus. An example of such a specific antibody is an IgA antibody. It is known that when IgA antibodies are produced in the respiratory tract and nasal cavity of humans, they act directly on influenza viruses, thereby preventing infection of the respiratory mucosal epithelium.

In this specification, deterioration in immune function against influenza virus infection, which occurs as a result of administration of the anti-influenza drug as described above, is referred to as “deterioration in the acquired immune function”. The composition for suppressing deterioration of the acquired immune function of the present invention can suppress “deterioration in the acquired immune function” that occurs as a result of using the anti-influenza drug. In the other words, ingesting the composition for suppressing deterioration of the acquired immune function according to the present invention allows for increasing the production amount of an antibody specific to influenza virus (e.g., IgA antibody, IgG antibody, etc.) produced in vivo. In addition, even when anti-influenza drugs are used, ingesting the composition for suppressing deterioration of the acquired immune function of the present invention allows for preventing a decrease in the production amount of the antibody specific to influenza virus produced in vivo.

Thus, administering the composition for suppressing deterioration of the acquired immune function according to the present invention together with, for example, an anti-influenza drug prevents deterioration of the immune function against influenza virus infection. This reduces the possibility that influenza patients who have used anti-influenza drugs are re-infected with influenza in the next season etc.

As shown in Examples described later, Bulgaricus bacterium R-1 strain also has a function of suppressing infection with influenza virus. In other words, it is preferable that the composition for suppressing deterioration of the acquired immune function of the present invention further has an infection inhibitory action against influenza virus. This makes it possible to prevent influenza infection as well by periodically (for example, every day) ingesting the composition for suppressing deterioration of the acquired immune function of the present invention.

As described above, in order to enhance the resistance to influenza virus and to suppress deterioration of the acquired immune function, it is preferable that the composition for suppressing deterioration of the acquired immune function of the present invention is periodically (preferably, everyday) ingested. To easily ingest the composition for suppressing deterioration of the acquired immune function of the present invention daily, it is preferable that the composition for suppressing deterioration of the acquired immune function of the present invention is in the form of fermented milk (for example, yoghurt). Yoghurt is widely eaten because of its good taste and advantages in beauty and health. Providing the composition for suppressing deterioration of the acquired immune function according to the present invention in the form of yoghurt makes it possible to comfortably ingest the required amount thereof every day.

For example, for fermented milk (e.g., a drink type) having a nonfat milk solid content of 8.0 wt %, an amount suitable for a single intake of the composition for suppressing deterioration of the acquired immune function of the present invention is preferably within a range from 50 mL to 200 mL inclusive, more preferably within a range from 80 mL to 150 mL inclusive, and even more preferably within a range from 100 mL to 120 mL inclusive. Alternatively, for example, for fermented milk (e.g., hard type or soft type) having a nonfat milk solid content of 8.0 wt %, an amount suitable for a single intake of the composition for suppressing deterioration of the acquired immune function of the present invention is preferably within a range from 50 g to 200 g inclusive, more preferably within a range from 80 g to 150 g inclusive, and even more preferably within a range from 100 g to 120 g inclusive. The frequency of intake is preferably more than or equal to 0.5 times a day and less than or equal to 5 times a day, more preferably more than or equal to one time a day and less than or equal to three times a day, and even more preferably more than or equal to one time a day and less than or equal to two times a day.

In addition, the composition for suppressing deterioration of the acquired immune function according to the present invention has physiologically active function of suppressing deterioration of acquired immune function due to use of an anti-influenza drug. Thus, it can be used as an active ingredient of foods and beverages (excluding animals and plants themselves), functional foods, functional drinks, medicines and the like. In other words, the foods and drinks (excluding animals and plants themselves), functional foods, functional drinks, medicinal products containing the composition for suppressing deterioration of the acquired immune function as an active ingredient are also included in the technical scope of the present invention.

In addition, the composition itself for suppressing deterioration of the acquired immune function of the present invention may be provided as foods and drinks (excluding animals and plants per se), functional foods, functional drinks, medicines, and the like. In other words, foods and drinks, functional foods, functional drinks, and the medicines according to another aspect of the present invention include, as the active ingredient, the lactic acid bacteria product provided by any one of the above-mentioned lactic acid bacteria of the genus Lactobacillus. And, it suppresses the deterioration of the acquired immune function due to using anti-influenza medicines.

In addition, in a case when the composition for suppressing deterioration of the acquired immune function according to the present invention is provided as foods or drinks, it is preferable to take the form of fermented milk from the viewpoints of production efficiency, ease of ingestion, palatability and the like. In one embodiment of the present invention, the fermented milk is a yoghurt obtained by adding lactic acid bacteria of the genus Lactobacillus to a milk material and then fermenting (culturing) the lactic acid bacteria.

The foods and drinks of the present invention may contain well-known additives that can be contained in foods (e.g., functional foods) besides the composition for suppressing deterioration of the acquired immune function. Examples of such additives include water, sugars, sugar alcohols, starch and processed starch, dietary fiber, cow milk, processed milk, soy milk, fruit juice, vegetable juice, fruits and vegetables and processed products thereof, proteins, peptides, amino acids, animal and plant herbal extracts, natural polymers (collagen, hyaluronic acid, chondroitin or the like), vitamins, minerals, thickeners, emulsifiers, preservatives, colorants, perfumes and the like.

In addition, in the case of using the composition for suppressing deterioration of the acquired immune function according to the present invention for medicines, well-known additives that can be contained in medicines may be contained besides the products of lactic acid bacteria. Examples of such additives include excipients, disintegrants, binders, fluidizing agents, corrigents, perfumes, colorants, sweeteners, solvents, oils and fats, thickeners, surfactants, gelling agents, stabilizers, preservatives, buffers, suspending agents, thickening agents and the like.

(2) Method for Producing Composition for Suppressing Deterioration of Acquired Immune Function

Next, a method for producing a composition for suppressing deterioration of the acquired immune function according to the present invention will be described. The method for producing a composition for suppressing deterioration of the acquired immune function according to the present invention includes a step of supplying a milk raw material to lactic acid bacteria of the genus Lactobacillus. For the lactic acid bacteria of the genus Lactobacillus to be used, those described in the above (1) can be adopted.

Examples of the milk raw material include animal milk such as cow milk and processed products thereof (e.g., skim milk, whole milk powder, skim milk powder, condensed milk, casein, whey, fresh cream, compound cream, butter, buttermilk powder, cheese, or the like), vegetable milk such as soymilk derived soybean and the like. Note that the milk raw material may be sterilized or may not be sterilized. In addition, various additives can be added to the milk raw material used for manufacturing the composition for suppressing deterioration of the acquired immune function.

Supplying milk raw material to lactic acid bacteria of the genus Lactobacillus and then fermenting or culturing lactic acid bacteria of the genus Lactobacillus makes it possible to produce lactic acid bacterial products which are to be a main component. The composition for suppressing deterioration of the acquired immune function produced using the production method according to the present invention may be obtained as fermented milk. In this case, the production method according to the present invention can also be described as a method of producing fermented milk having the function of suppressing deterioration of the acquired immunity by supplying milk raw material to lactic acid bacteria of genus Lactobacillus.

The raw material used for producing this fermented milk may contain not only the above-described milk raw material but also various other components. Accordingly, as a raw material used for producing fermented milk, for example, what is called a fermented milk raw material mix can be used. A fermented milk raw material mix is a mixture containing raw milk and other ingredients. This fermented milk raw material mix is obtained by heating, dissolving, and mixing raw materials commonly used for producing fermented milk such as milk raw material, water, other optional ingredients (e.g., sugar, saccharides, sweetener, acidulant, mineral, vitamin, perfume or the like), and the like. Milk raw materials may include raw milk, pasteurized milk, skimmed milk, whole milk powder, skimmed milk powder, full fat concentrated milk, defatted concentrated milk, butter milk, butter, cream, cheese and the like. In addition, whey protein concentrate (WPC), whey protein isolate (WPI), a-lactalbumin (α-La), β-lactoglobulin (β-Lg) and the like may be contained in the milk raw material.

As in the conventional method, fermented milk is produced through steps such as a step of preparing a raw material mix, a step of (heating) sterilizing the raw material mix, a step of cooling the raw material mix, a step of adding a starter, a step of fermentation, a step of cooling fermented milk and the like. In the step of preparing the raw material mix, raw materials are mixed (blended). Note that in the above process, ordinary conditions used for producing fermented milk may be appropriately adopted. In addition, it is preferable that the step of (heating) sterilizing the raw material mix, the step of cooling the raw material mix, the step of adding the starter, the step of fermentation and the step of cooling the fermented milk are performed in this order.

As a medium for culturing lactic acid bacteria, a commonly used medium can be used. In other words, any medium can be used as long as it contains, to the appropriate extent, a main carbon source as well as a nitrogen source, inorganic matter and other nutrients. As the carbon source, lactose, glucose, sucrose, fructose, starch hydrolyzate, blackstrap molasses or the like can be used depending on the assimilability of the used bacteria. As the nitrogen source, organic nitrogen-containing substances such as casein hydrolyzate, whey protein hydrolyzate, α-lactalbumin, β-lactoglobulin, glycomacropeptide, soy protein hydrolyzate and the like can be used. In addition, meat extract, fish meat extract, yeast extract and the like can be used as a growth promoting agent.

Lactic acid bacteria are preferably cultured in an anaerobic state; it is usually preferable to culture in a microaerophilic state used for liquid static culture or the like. Note that as a culturing method under an anaerobic condition, a known method such as a method of culturing under a carbon gas phase can be adopted; other methods may be adopted. In general, the culture temperature is preferably within a range from 30° C. to 47° C. inclusive, more preferably within a range from 35° C. to 46° C. inclusive, and even more preferably within a range from 37° C. to 45° C. inclusive. The pH of the medium while lactic acid bacteria is being cultured is preferably maintained within a range of 6 to 7 inclusive; other pH ranges may be used as long as the bacterium grows in the determined range of pH. The culture time of lactic acid bacteria or the like is usually preferably within a range from one hour to 48 hours inclusive, more preferably within a range from 8 hours to 36 hours inclusive, and even more preferably within a range from 10 hours to 24 hours inclusive.

Fermented milk typically has a non-fat milk solid content of 8 wt % or more, and the number of lactic acid bacteria or yeast number is typically within a range from 10⁶/mL to 10¹¹/mL inclusive.

The production method of the present invention as described above makes it possible to produce a composition for suppressing deterioration of the acquired immune function due to the use of an anti-influenza drug. Note that, the composition for suppressing deterioration of the acquired immune function described in the above (1) is an example of a composition for suppressing deterioration of the acquired immune function produced by the production method of the present invention.

It should be considered that the embodiment disclosed in this specification is merely an example in all respects and it should not be restrictive. The scope of the present invention is defined not by the above description but by the scope of the claims, and it is intended that all modifications within meaning and scope equivalent to the claims are included.

### EXAMPLE

Hereinafter, the present invention will be described in more detail with reference to examples. The following examples are merely illustrative, and should not limit the present invention.

Test 1

In Test 1, the influence on the amount of anti-influenza-specific antibody (IgA antibody and IgG antibody) by ingesting yoghurt (hereinafter referred to as “R-1 yoghurt”) produced using Bulgaricus bacterium R-1 strain was examined. More specifically, it was examined whether there was a difference in the amount of antibody produced after slight infection with influenza virus between a mouse to which R-1 yoghurt was previously administered and a mouse to which R-1 yoghurt was not administered.

(1-1) Producing R-1 Yoghurt Yoghurt was produced by adding Lactobacillus delbrueckii subsp. bulgaricus OLL 1073R-1 bacterium (Deposit number: FERM BP-10741) (hereinafter referred to as “Bulgaricus bacterium R-1 strain”) and streptococcus thermophilus, as a starter, to a mixture containing raw milk, skimmed milk powder, cream, sugar and stevia, and then fermenting them.

(1-2) Administration of R-1 Yoghurt and the Like to Mice

As a test subject, 6-week old female BALB/c mice (Japan SLC Co., Ltd.) were used. In testing, mice were grouped into the following four groups. Nine or ten mice were used for each group.
MC: control group to which ultrapure water (substitute for R-1 yoghurt) and 0.5% methylcellulose solution were administered (control group for R1 group).
R1: group to which R-1 yoghurt and 0.5% methyl cellulose solution were administered.
OSV: group to which ultrapure water and oseltamivir dissolved in 0.5% methylcellulose solution were administered.
OSV+R1: group to which R-1 yoghurt and oseltamivir dissolved in 0.5% methylcellulose solution were administered.

As described above, in the MC group and the OSV group, ultrapure water was used as a comparative control for R-1 yoghurt. In the MC group and R1 group, a 0.5% methylcellulose solution (0.5 w/v % Methyl Cellulose 400) (Wako Pure Chemical Industries, Ltd.), which is a dissolution liquid, was used as a comparative control for OSV.

Among the above four groups, ultrapure water (a substitute for R-1 yoghurt) was orally administered to mice of MC group and OSV group for 21 days (3 weeks) before infection with influenza virus. A single dose was 0.4 mL. The number of administrations was once a day, and the administration was continued for 14 days even after the virus infection.

R-1 yoghurt was orally administered, for 21 days (3 weeks) before infection with influenza virus, to R1 group and OSV +R1 group mice out of the above four groups. A single dose of R-1 yoghurt was 0.4 mL. The number of administrations was once a day, and the administration was continued for 14 days even after the virus infection.

(1-3) Influenza Virus Infection and OSV Administration to Mice

Influenza viruses were transnasally infected with 0.5 pfu (plaque-forming unit)/mouse to the mice of each group in the above (1-2). The influenza virus used is influenza A virus (IAV)/Puerto Rico/8/1934 (PR 8) (H1N1) (hereinafter abbreviated as PR 8).

After viral infection, 0.5% methylcellulose (not containing OSV), which is a solvent for OSV, was orally administered to mice of MC group and R1 group. A single dose of methylcellulose was 0.1 mL. The number of administrations was twice a day, and the number of administration days was 14 days.

After virus infection, oseltamivir (phosphate) (Funakoshi Co., Ltd.) which is one type of anti-influenza virus drug was dissolved in 0.5% methyl cellulose and then was administered orally to mice of the OSV group and mice of the OSV+R1 group. A single dose of oseltamivir (phosphate) was 0.1 mg/0.1 mL/mouse. The number of administrations was twice a day, and the number of administration days was 14 days.

As described above, anti-influenza drug (OSV) was administered to mice of OSV group and OSV+R1 group, and no anti-influenza drug was administered to mice of MC group and R1 group.

(1-4) Evaluation of Anti-Influenza Specific Antibody Titer by ELISA

Anti-influenza specific antibody titer was evaluated by ELISA on the fourteenth day after virus infection. More specifically, the antibody titer of IgA in the lung lavage fluid of mice and the antibody titer of IgG in serum of mice were evaluated.

The ELISA was performed in the following procedure. An antigen preparation liquid (PR8 (0.5 μg/ml) BSA (0.1%)/PBS) was added in a 96-well plate at 100 μL/well to immobilize the antigen (antigen 0.05 μg/well of antigen). After holding at 4° C. for 12 hours, each well was washed three times with washing buffer (50 mM Tris-HCl (pH 8.0), 0.14M NaCl, 0.05% Tween 20). A sufficient amount of blocking buffer (50 mM Tris-HCl (pH 8.0), 0.14M NaCl, 1% BSA) was added to each well and kept at 37° C. for 2 hours. After each well was washed three times with a washing buffer, a measurement sample (Lung lavage fluid or serum collected from mice of each group) diluted appropriately with a sample buffer (50 mM Tris-HCl (pH 8.0), 0.14M NaCl, 0.05% Tween 20, 1% BSA) was added at 100 μL/well. Each well was washed 5 times with wash buffer.

Sufficient amount of 10,000-fold diluted HRP-conjugate anti-mouse IgG (#A90-131P, manufactured by BETHYL LABORATORIES) or 2,000-fold diluted HRP-conjugate anti-mouse IgA (#A90-103P, manufactured by BETHYL LABORATORIES) was added to each well. Thereafter, each well was washed five times with a washing buffer. Chromogenic solution (TMB (3, 3′, 5, 5′-tetramethylbenzidine), manufactured by KPL, SureBlue, #52-00-02) was added at 100 μL/well, and was left at room temperature for 15 minutes. Subsequently, stop solution (TMB Stop Solution, manufactured by KPL, #50-85-05) was added to it at 100 μL/well. Thereafter, the absorbance at 450 nm was measured for each measurement sample, and the anti-influenza specific antibody titer in the measurement sample was evaluated.

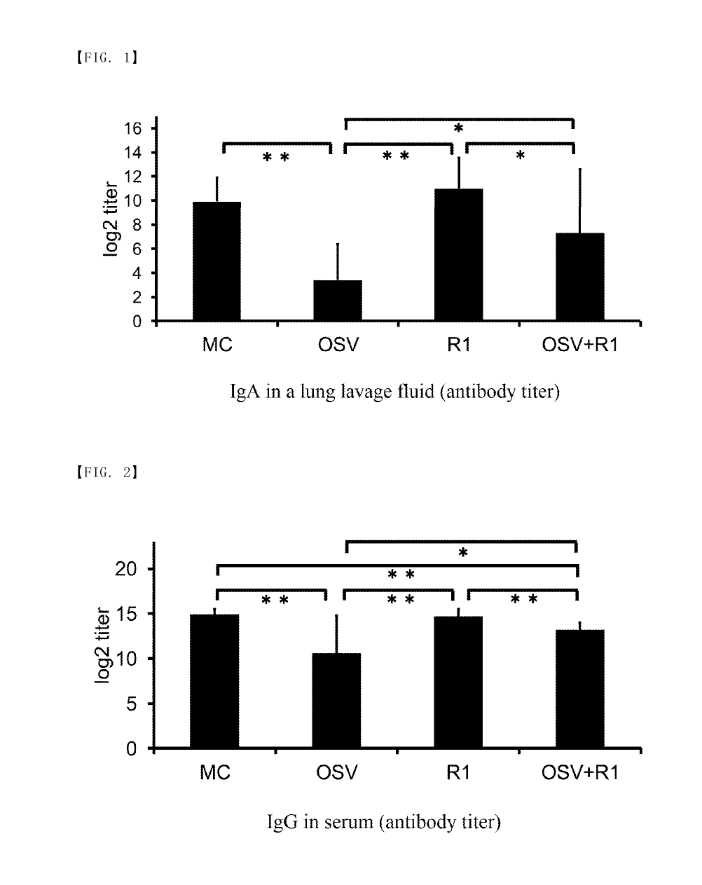

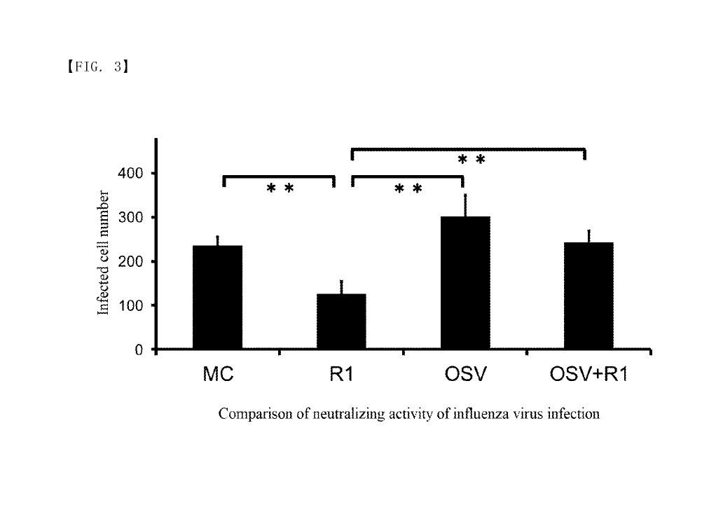

FIG. 1 shows the measurement results of IgA. FIG. 2 shows the measurement results of IgG. In graphs of each figure, each vertical bar indicates the standard deviation in its corresponding group (MC group, OSV group, R1 group, OSV+R1 group). In addition, the mark * between groups indicates that a significant difference exists at a risk rate of less than 5%, and the mark ** indicates that a significant difference exists at a risk ratio of less than 1%.

As shown in FIG. 1, the amount of IgA antibody in the lung lavage fluid was significantly increased in the group to which R-1 yoghurt and oseltamivir (OSV+R1 group) was administered as compared with the group to which only oseltamivir (OSV group) was administered. In addition, as shown in FIG. 2, the amount of IgG antibody in the serum was significantly increased in the OSV+R1 group as compared with the OSV group.

Based on the above results, it was confirmed that R-1 yoghurt prepared using Bulgaricus bacterium R-1 strain (Lactobacillus delbrueckii subsp. bulgaricus OLL 1073 R-1 bacterium) had the effect for suppressing deterioration of the acquired immune function due to anti-influenza drugs.

Test 2

In Test 2, the nasal cavity lavage fluid was collected from mice of each group (MC group, OSV group, R1 group, OSV+R1 group) obtained in (1-3) of Test 1 above. 50 μL of this nasal cavity lavage fluid was neutralized with influenza virus PR8 (100 pfu). Subsequently, it was acted on MDCK cells (canine kidney-derived cells), and the number of infected cells was counted 16 hours later, thereby evaluating the neutralizing activity of influenza virus infection of R-1 yoghurt.

The results are shown in FIG. 3. In the graph, each vertical bar indicates the standard deviation in its corresponding group (MC group, OSV group, R1 group, OSV+R1 group). In addition, the mark * between groups indicates that a significant difference exists at a risk rate of less than 5%, and the mark ** indicates that a significant difference exists at a risk ratio of less than 1%. In the R-1 yoghurt administered group (R1 group), a significant decrease in infected cell number was observed as compared with the control group (MC group). There was no significant difference between R-1 yoghurt and OSV administered group (OSV+R1 group) and OSV administered group (OSV group); as shown in FIG. 3, the number of infected cells tended to decrease slightly toward that of OSV+R1 group as compared with the OSV group.

Based on the above results, it was confirmed that R-1 yoghurt prepared using Bulgaricus bacterium R-1 strain (Lactobacillus delbrueckii subsp. bulgaricus OLL 1073 R-1 bacterium) had the effect for enhancing the neutralizing activity against influenza virus.

Deposit number

FERM BP-10741

1. A composition for suppressing deterioration of an acquired immune function which contains a lactic acid bacterial product by lactic acid bacteria of the genus Lactobacillus as an active ingredient and suppresses deterioration of the acquired immune function due to the use of an anti-influenza drug.

2. The composition for suppressing deterioration of the acquired immune function according to claim 1, wherein the lactic acid bacteria of the genus Lactobacillus is classified as a subspecies bulgaricus.

3. The composition for suppressing deterioration of the acquired immune function according to claim 1, wherein the lactic acid bacteria of the genus Lactobacillus is Lactobacillus delbrueckii subsp. bulgaricus.

4. The composition for suppressing deterioration of the acquired immune function according to claim 1, which is fermented milk.

5. The composition for suppressing deterioration of the acquired immune function according to claim 1, which further has an infection inhibitory action against influenza virus.

6. A method for producing a composition for suppressing deterioration of an acquired immune function for supplying a milk raw material to a lactic acid bacterium of the genus Lactobacillus and suppressing deterioration of the acquired immune function due to the use of an anti-influenza drug.
